(19) United States
(12) Patent Application Publication
(10) Pub. No.: US 2024/0417690 A1
(43) Pub. Date: Dec. 19, 2024

(54) CELL POPULATION OF HUMAN URINE-DERIVED CELLS, AND CELL POPULATION OF MYOTUBES INDUCED THEREFROM AND PRODUCTION METHOD THEREFOR

(71) Applicants: NIPPON SHINYAKU CO., LTD., Kyoto-shi (JP); NATIONAL CENTER OF NEUROLOGY AND PSYCHIATRY, Tokyo (JP)

(72) Inventors: Yoshitsugu AOKI, Tokyo (JP); Katsuhiko KUNITAKE, Tokyo (JP)

(73) Assignees: NIPPON SHINYAKU CO., LTD., Kyoto-shi (JP); NATIONAL CENTER OF NEUROLOGY AND PSYCHIATRY, Tokyo (JP)

(21) Appl. No.: 18/745,735

(22) Filed: Jun. 17, 2024

(30) Foreign Application Priority Data
Jun. 19, 2023 (JP) 2023-100081

(51) Int. Cl. C12N 5/077; C12N 5/071

(52) U.S. Cl. CPC C12N 5/0658; C12N 5/0684; C12N 2506/25; C12N 2510/00

(57) ABSTRACT

Disclosed are a cell population of human urine-derived cells positive for CD90, a cell population of myotubes induced from the cell population of the human urine-derived cells positive for CD90, and a method for producing a myotube derived from the human urine-derived cell, comprising a step of separating the human urine-derived cells positive for CD90.

All patent and non-patent literatures cited herein are incorporated herein by reference in their entirety.

### TECHNICAL FIELD

The present disclosure relates to a cell population of human urine-derived cells positive for CD90, a population of myotubes induced from the cell population, and a production method for the population of the myotubes.

### BACKGROUND

Human urine-derived cells (UDCs) are primary cultured cells derived from an upper urinary tract, which can be collected from human urine. A cell population of human urine-derived cells is known to be a heterogeneous cell population including cells of various forms and origins, such as renal epithelial cells and urothelial cells. In addition, although human urine-derived cells are human-derived primary cultured cells, the cells have an excellent proliferative capacity and can be collected from urine, which is a noninvasively obtainable specimen; therefore, approaches have been taken that attempt to induce human urine-derived cells into various cells to use as medical and biological tools (Non Patent Literature 1).

One example of the cells in which the induction from human urine-derived cells has been studied includes myotubes. Myotubes (also referred to as “myotube cells”) are juvenile muscle cells. Since collection of myotubes from a human subject is highly invasive, induction of myotubes from human urine-derived cells has been attempted, for the purpose of noninvasively obtaining myotubes for use in drug evaluation of muscular diseases or for use in the study of muscular diseases or as a biopsy (Non Patent Literature 2).

The present inventors have developed a method for producing a myotube (myotube cell) from urine-derived cells, comprising a step of introducing the myoblast determination protein 1 (MyoD1), which is a muscle regulatory factor (MRF), into human urine-derived cells (urinary cells) and a step of exposing the urine-derived cells to at least one epigenetic control compound (Patent Literature 1).

### CITATION LIST

### Patent Literature

Patent Literature 1: International Publication No. 2020/136696.

### Non Patent Literature

Non Patent Literature 1: Maria Sofia Falzarano and Alessandra Ferlini, “Urinary Stem Cells as Tools to Study Genetic Disease: Overview of the Literature”, J. Clin. Med. 8 (5), 627 (2019).
Non Patent Literature 2: Ellis Y. Kim et al., “Direct reprogramming of urine-derived cells with inducible MyoD for modeling human muscle disease”, Skeletal Muscle volume 6:32 (2016).
Non Patent Literature 3: Ricardo Mondragon-Gonzalez and Rita C R Perlingeiro, “Recapitulating muscle disease phenotypes with myotonic dystrophy 1 induced pluripotent stem cells: a tool for disease modeling and drug discovery”, Dis. Model Mech. 11 (7): dmm034728 (2018). Non Patent Literature 4: Ting Zhou et al., “Generation of human induced pluripotent stem cells from urine samples”, Nature Protocols 7, 2080-2089 (2012).

### SUMMARY

Since a cell population of human urine-derived cells is a heterogeneous cell population, it can be considered that the cell population includes cells which are easy to be differentiated into myotubes (their muscle differentiation potential is high) and those which are difficult to be differentiated into myotubes (their muscle differentiation potential is low). Accordingly, it can be considered that, if a population of cells of which muscle differentiation potential is high can be sorted from the cell population of the human urine-derived cells, myotubes induced from human urine-derived cells can be efficiently produced.

In addition, it is known that the increase in the expression level of some genes or proteins specific to muscular diseases may be observed when the muscle differentiation proceeds and more mature myotubes are formed (Non Patent Literature 3). Accordingly, it can be effective to sort the population of the cells of which muscle differentiation potential is high, for the purpose of using the myotubes induced from the human urine-derived cells for drug evaluation of muscular diseases or for use in the study of muscular diseases or as a biopsy.

A cell surface marker specific to human urine-derived cells of which muscle differentiation potential is high has not been known to date.

An object of the present disclosure is to provide a cell population of human urine-derived cells of which muscle differentiation potential is high. Further, another object of the present disclosure is to provide a cell population of myotubes induced from the cell population of human urine-derived cells of which muscle differentiation potential is high, and a production method thereof.

The present inventors have found that a mesenchymal stem cell maker, CD90 (cluster of differentiation 90; also known as THY1) is a cell surface marker specific to human urine-derived cells of which muscle differentiation potential is high, and completed the present disclosure.

The present disclosure relates to, for example, the following.
[1] A cell population of human urine-derived cells positive for CD90.
[2] The cell population according to [1], for use in induction of a myotube.
[3] The cell population according to [1] or [2], in which the human urine-derived cells positive for CD90 are derived from a muscular disease patient or a muscular dystrophy patient.
[4] A method for producing a myotube derived from a human urine-derived cell, comprising a step of separating a human urine-derived cell positive for CD90.
[5] The method according to [4], in which the step of separating the human urine-derived cell positive for CD90 is a step of separating a human urine-derived cell positive for CD90 from a human urine-derived cell prepared from urine of a muscular disease patient or a muscular dystrophy patient.
[6] The method according to [4] or [5], comprising a step of inducing a myotube from the separated human urine-derived cell positive for CD90 after the step of separating.
[7] The method according to [6], in which the step of inducing the myotube comprises introducing a MYOD1 gene into the human urine-derived cell positive for CD90.
[8] The method according to [6] or [7], in which the step of inducing the myotube does not comprise making an induced pluripotent stem cell from the human urine-derived cell positive for CD90.
[9] A cell population of myotubes induced from a cell population of human urine-derived cells positive for CD90.
[10] The cell population according to [9], in which the human urine-derived cell positive for CD90 is derived from a muscular disease patient or a muscular dystrophy patient.
[11] A method for separating a human urine-derived cell of which muscle differentiation potential is high, comprising a step of separating a human urine-derived cell positive for CD90.
[12] The method according to [11], in which the human urine-derived cell is derived from a muscular disease patient or a muscular dystrophy patient.
[13] A kit comprising: an anti-CD90 antibody or an antigen-binding fragment thereof; and a package insert in which there is described that a CD90 expression in a human urine-derived cell is evaluated, and that the human urine-derived cell that is evaluated as the amount of CD90 expressed is high is separated as a human urine-derived cell of which muscle differentiation potential is high.
[14] A method for evaluating muscle differentiation potential of a human urine-derived cell, comprising a step of evaluating the CD90 expression in the human urine-derived cell.
[15] The method according to [14], in which the human urine-derived cell is a human urine-derived cell derived from a muscular disease patient or a muscular dystrophy patient.
[16] A kit comprising: an anti-CD90 antibody or an antigen-binding fragment thereof; and a package insert in which there is described that the CD90 expression in a human urine-derived cell is evaluated, and that the human urine-derived cell that is evaluated as the amount of CD90 expressed is high is evaluated as a human urine-derived cell of which muscle differentiation potential is high.

According to one embodiment of the present disclosure, a cell population of human urine-derived cells of which muscle differentiation potential is high can be provided.

According to one embodiment of the present disclosure, a cell population of myotubes derived from a cell population of a human urine-derived cell of which muscle differentiation potential is high, and a production method therefor can be provided.

According to the cell population of the human urine-derived cells in one embodiment of the present disclosure, a cell population of myotubes can be highly efficiently produced.

According to the cell population of the human urine-derived cells in one embodiment of the present disclosure, a cell population of myotubes can be produced, while suppressing the impact of the heterogenicity of the human urine-derived cells of each subject whose urine has been collected.

According to one embodiment of the present disclosure, it is possible to provide a cell population of human urine-derived cells, which can be induced into myotubes, though not involving producing induced pluripotent stem cells from human urine-derived cells.

According to one embodiment of the present disclosure, it is possible to produce a cell population of myotubes that have been induced from a cell population of human urine-derived cells, though not involving producing induced pluripotent stem cells from human urine-derived cells.

According to one embodiment of the present disclosure, it is possible to provide a cell population of human urine-derived cells, which can be induced into myotubes, though not involving exposing to an epigenetic control compound.

According to one embodiment of the present disclosure, it is possible to produce a cell population of myotubes derived from a cell population of a human urine-derived cells, though not involving exposing to an epigenetic control compound.

### BRIEF DESCRIPTION OF THE DRAWINGS

FIG. 1 shows a schematic diagram depicting a design of a retroviral vector (MYOD1 viral vector) that was used in the introduction of a MYOD1 gene into a human urine-derived cell in Experimental Method 2;

FIG. 2 shows a diagram depicting single cell RNAseq analysis results of a cell population of which muscle differentiation potential is high (Good UDCs) and a cell population of which muscle differentiation potential is low (Bad UDCs) before and after the muscle differentiation induction, in Example 1;

FIG. 3 shows a graph depicting fluorescence intensity distribution in separating a cell population of human urine-derived cells positive for CD90 by flow cytometry method in Example 2;

FIG. 4 shows a photographs depicting fluorescence images of the cell population obtained by inducing the muscle differentiation from a cell population of human urine-derived cells negative for CD90 or positive for CD90 in Example 3;

FIG. 5 shows a graph depicting the fusion index of the cell population obtained by inducing the muscle differentiation from the cell population of the human urine-derived cells negative for CD90 or positive for CD90 in Example 3;

FIG. 6 shows photographs depicting Western blotting results of the cell population obtained by inducing the muscle differentiation from the cell population of the human urine-derived cells negative for CD90 or positive for CD90 in Example 3;

FIG. 7 shows a graph depicting the band intensity of MYOD1 standardized with the band intensity of a-tubulin (a-Tub) of FIG. 6 in Example 3;

FIG. 8 shows a graph depicting the band intensity of MYHC standardized with the band intensity of a-tubulin (a-Tub) of FIG. 6 in Example 3;

FIG. 9 shows a diagram depicting Western blotting results when adding an exon skipping agent to the cell population of myotubes induced from the cell population of the human urine-derived cells positive for CD90 derived from a DMD patient in Example 4; and

FIG. 10 shows a graph depicting the band intensity of DYS1 standardized with the band intensity of a-tubulin (a-Tub) of FIG. 9 in Example 4.

### DETAILED DESCRIPTION

Hereinafter, embodiments of the present disclosure will be described; however, the present disclosure is not limited to the following embodiments.

A first aspect of the present disclosure is a cell population of human urine-derived cells positive for CD90.

Human urine-derived cells (UDCs) are primary cultured cells derived from an upper urinary tract, which can be collected from human urine. A cell population of human urine-derived cells are known to be a heterogeneous cell population including cells of various forms and origins, such as renal epithelial cells and urothelial cells. The human urine-derived cells according to the present disclosure may be, for example, cells obtained by isolating urine collected from a human subject or a culture thereof, or cells obtained by culturing cells obtained by isolating urine collected from a human subject. A method for isolating cells in this case is not particularly limited, as long as it is a method whereby the cells can be separated from urine including the cells or a culture thereof, and for example, it may be centrifugation or filtration, and preferably centrifugation.

A method for preparing human urine-derived cells is known in the art (e.g., Non Patent Literature 4). The method for preparing human urine-derived cells according to the present disclosure is not particularly limited, and for example, preparation can be performed by the method shown below. After centrifuging urine collected from a human subject and removing the supernatant, pellets are mixed with a primary culture medium and incubated at about 37° C., and then the mixture is cultured in a growth medium to select cells colonized approximately several days to 2 weeks after the culture initiation. The cells thus obtained become stable cell line having similar features even after culture passaging several times (Non Patent Literature 4).

The human urine-derived cells according to the present disclosure may be any as long as they are derived from urine collected from a human subject, and for example, may be derived from urine collected from a healthy individual, derived from urine collected from a muscular disease patient or a muscular dystrophy patient (e.g., Duchenne muscular dystrophy patient), or a mixture of cells derived from both; and these are freely selectable according to the purpose by those skilled in the art. In addition, the human urine-derived cells according to the present disclosure may be cells derived from urine obtained by one collection from a human subject, or a mixture of cells derived from urine obtained by several collections from a human subject.

Examples of the urine collected from a muscular dystrophy patient may include urine collected from a patient of Duchenne muscular dystrophy, Becker muscular dystrophy, Fukuyama muscular dystrophy, or congenital muscular dystrophy such as merosin-deficiency or Ullrich syndrome. Examples of the urine collected from a muscular disease patient may include urine collected from a patient of neuromuscular junction diseases such as myopathy, inflammatory muscle disease, and myasthenic syndrome, neurodegenerative diseases such as amyotrophic lateral sclerosis, peripheral nervous system disorders such as spinal muscular atrophy, diseases that lead to disuse muscle atrophy including stroke sequela, sarcopenia or cancer cachexia.

In the present disclosure, a cell population means a group (set, or mass) consisting of a plurality of cells. In other words, the cell population according to the present disclosure is not limited to a physical aggregate of cells, representative examples of which are colonies or spheroids, but includes a group consisting of a plurality of cells in a state of being suspended in a solution. The cell population according to the present disclosure may be a group consisting of a plurality of cells suspended in one liquid, the cells being spatially sequentially present, or a group consisting of a plurality of cells that are present in one physically partitioned container (e.g., test tube, tube, cell culturing container, or culture dish). The number of the cells composing the cell population according to the present disclosure may be, for example, 2 or more, 10 or more, 100 or more, 1000 or more, or 10000 or more.

The cell population according to the first aspect of the present disclosure is a cell population of human urine-derived cells positive for CD90.

CD90 (cluster of differentiation 90) is a cell surface protein, also known as THY1. In humans, CD90 is known to be expressed in a wide variety of organs including muscles, brain, and kidney. Also, CD90 is known to be one of mesenchymal stem cell markers.

In the present disclosure, a human urine-derived cell positive for CD90 is a human urine-derived cell of which expression level of CD90 is high. In addition, a human urine-derived cell positive for CD90 can be said to be a human urine-derived cell that highly expresses CD90. The expression level or expression amount of CD90 herein may be an expression level or expression amount evaluated according to a method conventionally used by those skilled in the art when evaluating the expression level or expression amount of cell surface proteins. Examples the evaluation method used in such an evaluation can include an immunostaining method by using labeled anti-CD90 antibodies, a Western blotting method and a quantitative PCR method (q-PCR method), and an immunostaining method by using labeled anti-CD90 antibodies or a Western blotting method is preferable from the viewpoint of enabling direct detection of proteins. More specifically, the expression level or expression amount of CD90 may be evaluated by, for example, using, as an index, the fluorescence intensity of the wavelength corresponding to the fluorescence wavelength of fluorochrome in cells when bringing anti-CD90 antibodies labeled by the fluorochrome into contact with the cells.

Specifically, a human urine-derived cell positive for CD90 may be, for example, a human urine-derived cell that meets at least one of the following (a) to (e). In one preferred embodiment, the expression level or expression amount of CD90 in the following (a) to (e) may be the fluorescence intensity of the wavelength corresponding to the fluorescence wavelength of fluorochrome when bringing anti-CD90 antibodies labeled by the fluorochrome into contact.
(a) A human urine-derived cell in which the expression level or expression amount of CD90 is 3 times or more, 5 times or more, or 10 times or more of the average value of the expression level or expression amount of CD90 in the human urine-derived cell prepared from the same human subject by the same method.
(b) A human urine-derived cell in which the expression level or expression amount of CD90 is higher than a lower limit of 80% confidence interval, 85% confidence interval, 90% confidence interval, 95% confidence interval, 98% confidence interval, or 99% confidence interval for the distribution, whichever average of the expression level or expression amount is larger, when fitting the expression level or expression amount of CD90 in the human urine-derived cell prepared from the same human subject by the same method, as superposition of 2 groups of normal distributions.
(c) A human urine-derived cell in which the expression level or expression amount of CD90 corresponds to top 10%, top 15%, top 20%, top 25%, top 30% or top 50% of the expression level or expression amount of CD90 in the human urine-derived cell prepared from the same human subject by the same method.
(d) A human urine-derived cell, in which the expression level or expression amount of CD90 is 3 times or more, 5 times or more, or 10 times or more of the average value of the expression level or expression amount of CD90 in the cell obtained by knockdown or knockout of CD90 in the human urine-derived cell.
(e) A human urine-derived cell in which the expression level or expression amount of CD90 is higher than an upper limit of 80% confidence interval, 85% confidence interval, 90% confidence interval, 95% confidence interval, 98% confidence interval, or 99% confidence interval, when fitting the distribution of the expression level or expression amount of CD90 in established cultured cell line of low CD90 expression (e.g., HT-29 cells, Caco-2 cells, HepG2 cells, or MDA-MB-231 cells), as normal distributions.

In one preferred embodiment, a human urine-derived cell positive for CD90 may be a human urine-derived cell that meets at least one of the following (a′) to (e′). In the following (a′) to (e′), “CD90 staining intensity” means the fluorescence intensity of the wavelength corresponding to the fluorescence wavelength of fluorochrome when bringing into contact with anti-CD90 antibodies labeled by the fluorochrome, and the fluorescence intensity may be measured by, for example, a flow cytometer.
(a′) A human urine-derived cell in which the CD90 staining intensity is 5 times or more of the average value of the CD90 staining intensity in the human urine-derived cell prepared from the same human subject by the same method.
(b′) A human urine-derived cell in which the CD90 staining intensity is higher than a lower limit of 95% confidence interval for the distribution, whichever CD90 staining intensity is larger, when fitting the CD90 staining intensity in the human urine-derived cell prepared from the same human subject by the same method, as superposition of 2 groups of normal distributions.
(c′) A human urine-derived cell in which the CD90 staining intensity corresponds to top 25% of the CD90 staining intensity in the human urine-derived cell prepared from the same human subject by the same method.
(d′) A human urine-derived cell, in which the CD90 staining intensity is 5 times or more of the average value of the CD90 staining intensity in the cell obtained by knockout of CD90 in the human urine-derived cell.
(e′) A human urine-derived cell in which the CD90 staining intensity is higher than an upper limit of 95% confidence interval, when fitting the distribution of the CD90 staining intensity in HT-29 cells, as normal distributions.

According to the first aspect of the present disclosure, the percentage of human urine-derived cells positive for CD90 may be, for example, 50% or more, 60% or more, 70% or more, 75% or more, 80% or more, 85% or more, 90% or more, 95% or more, 98% or more, 99% or more, or 100% of the cells included in a cell population of human urine-derived cells positive for CD90. In one preferred embodiment, the percentage of human urine-derived cells positive for CD90 may be 15 90% or more of the cells included in a cell population of human urine-derived cells positive for CD90. The cell population according to the first aspect of the present disclosure may be a cell population obtained by sorting human urine-derived cells such that the human urine-derived cells positive for CD90 account for the above percentage.

20 The muscle differentiation potential of the cell population of human urine-derived cells positive for CD90 according to the first aspect of the present disclosure is high. In other words, the cell population of human urine-derived cells positive for CD90 according to the first aspect of the present disclosure is easy to differentiate into myotubes (easy to be induced). Therefore, in one embodiment, the cell population of human 25 urine-derived cells positive for CD90 according to the first aspect of the present disclosure may be a cell population to be used for induction in myotubes.

Myotubes (also referred to as “myotube cells”) are juvenile muscle cells. Myotubes are so-called multinucleated cells that has two or more nuclei, and myofibrils are formed at the periphery of myotubes. Accordingly, the fact that a cell is a myotube, or a cell has been induced in a myotube can be evaluated based on the fact that the cell has a characteristic form of such a myotube. Specifically, for example, whether a cell is a myotube or not can be evaluated by staining the cell nucleus with a nucleus staining reagent such as DAPI or Hoechst and based on the fact that the cell has two or more nuclei. Also, for example, whether a cell is a myotube or not can be evaluated based on the fact that a fluorescence image derived from a myofibril is observed when staining the cell with an antibody against a protein that is highly expressed in the myofibril, such as a labeled anti-myosin (MYHC) antibody, or the fact that the cell exhibits strong fluorescence when staining the cell with an antibody against a muscle-specific transcription factor, such as a labeled anti-myogenin antibody. Further, for example, whether a cell is a myotube or not can be evaluated based on the fact that the proportion of the cell nuclei contained in the myotube (fusion index) to the cell nuclei in the visual field for the plate-cultured cells is high.

According to the cell population of the first aspect of the present disclosure, it is possible to provide a cell population of human urine-derived cells of which muscle differentiation potential is high. Therefore, according to the cell population of the first aspect of the present disclosure, it is possible to efficiently produce a cell population of myotubes. When it is possible to efficiently produce a cell population of myotubes, the population is useful for high-throughput screening of a therapeutic agent and a prophylactic agent that target myotubes. Also, according to the cell population of the first aspect of the present disclosure, it is possible to induce human urine-derived cells in myotubes though not involving production of induced pluripotent stem cells from human urine-derived cells. Further, for example, according to the cell population of the first aspect of the present disclosure, it is possible to induce human urine-derived cells in myotubes, though not involving exposure to an epigenetic control compound.

In addition, according to the cell population of the first aspect of the present disclosure, it is possible to induce human urine-derived cells in myotubes to obtain a cell population of myotubes, while suppressing the influence of muscle differentiation potential varied by each collection of urine collected from a specific human subject. Therefore, for example, it is possible to obtain cell populations of myotubes induced from human urine-derived cells prepared from urine collected from one muscle disease patient or muscular dystrophy patient at a plurality of time points; and the cell populations of myotubes are useful for monitoring of the disease progression, or disease studies by acquisition of a gene profile and phenotype at each time point.

In addition, according to the cell population of the first aspect of the present disclosure, it is possible to induce human urine-derived cells in myotubes to obtain a cell population of myotubes, while suppressing the influence of muscle differentiation potential varied by human subjects. Therefore, for example, it is possible to obtain cell populations of myotubes from human urine-derived cells of individual muscle disease patients or muscular dystrophy patients; and the cell populations of myotubes are, for example, useful for tests on therapeutic agents and prophylactic agents useful for individual patients without actual administration (so-called companion diagnostics), or disease studies by acquisition of gene profiles and phenotypes of the myotubes derived from the individual patients.

One example of specific application can include tests and companion diagnostics on exon skipping agents in human urine-derived cells derived from a muscular dystrophy patient, and more specifically, can include tests and companion diagnostics on exon skipping agents in human urine-derived cells derived from a Duchenne muscular dystrophy patient. Duchenne muscular dystrophy (DMD) is a chronic progressive orphan muscle disease in which dystrophin is missing from sarcolemma due to mutations in the responsible gene, DMD, and degeneration of muscle fibers occurs. An exon skip therapy is known to obtain a therapeutic method for Duchenne muscular dystrophy. The exon skip therapy is a therapy that targets a pre-mRNA, regulates splicing by an antisense nucleic acid pharmaceutical to convert frame-shift mutation to in-frame mutation, and aims to recover the expression of a truncated dystrophin protein. The present inventors have achieved in 2020 the practical application of viltolarsen (exon 53 skipping agent), which is an antisense nucleic acid pharmaceutical for Duchenne muscular dystrophy caused by exon 53 deletion mutation. On the other hand, in the course of extending targeted deletion mutation, it has been revealing that it is not necessarily possible to expect the exon skipping induction effect in the body of a patient administered by only the genomic DNA mutation pattern of the patient. Accordingly, since it is possible to obtain a cell population of myotubes from the human urine-derived cells of individual Duchenne muscular dystrophy patients according to the cell population of the first aspect of the present disclosure, it is possible to obtain profiles of personalized agents of which effectiveness is expected for the individual patients (so-called companion diagnostics), and it is expected to be possible to support providing medicine personalized for each patient (personalized medicine, and precision medicine).

A second aspect of the present disclosure is a method for producing a myotube derived from a human urine-derived cell, comprising a step of separating a human urine-derived cell positive for CD90 (separation step). As the human urine-derived cell positive for CD90 in the present aspect, it is possible to use the human urine-derived cell positive for CD90 in the cell population of the human urine-derived cell positive for CD90 of the first aspect of the present disclosure. In other words, in the separation step, the human urine-derived cells positive for CD90 described in the first aspect of the present disclosure is separated.

In other words, as the human urine-derived cells that are the origin of separation (i.e., human urine-derived cells before separation) in the separation step, the cells described in the first aspect of the present disclosure can be used, and for example, the cells prepared by the preparation method described in the first aspect can be used. In addition, in one embodiment of the second aspect, the separation step may be a step of separating human urine-derived cells positive for CD90 from the human urine-derived cells prepared from urine of a muscular disease patient or a muscular dystrophy patient.

The method for separating cells in the separation step is not particularly limited as long as it is a method whereby human urine-derived cells positive for CD90 can be separated. Examples of such a method can include a method that causes human urine-derived cells to be brought into contact with anti-CD90 antibodies labeled by fluorochrome, followed by separating cells of which fluorescence intensity is determined to be high by a flow cytometer as human urine-derived cells positive for CD90 (so-called flow cytometry method). Another example thereof can include a method that causes human urine-derived cells to be brought into contact with anti-CD90 antibodies labeled by biotin or an affinity tag such as anti-His-tag antibody, and then brought into contact with a bead, column or culture dish surface-modified by molecules recognizing streptavidin or the above affinity tag such as His-tag to trap human urine-derived cells positive for CD90 on the surface of the bead, column or culture dish, followed by adding biotin or His-tag, or a derivative thereof to release the human urine-derived cells positive for CD90 from the surface, thereby separating the cells (so-called affinity tag purification method or affinity chromatography method). In one preferred embodiment, the method for separating cells in the separation step may be a flow cytometry method.

The conditions in which human urine-derived cells are brought into contact with the labeled anti-CD90 antibodies in the separation methods are not particularly limited, and it is possible to perform the method under the conditions that those skilled in the art conventionally use. For example, it is possible to perform the method as follows: a solution is prepared by diluting about 11-fold to 10001-fold a commercially available solution of labeled anti-CD90 antibodies with a phosphate buffer solution (PBS) containing 2% fetal bovine serum, human urine-derived cells are exposed to the solution, and then incubated under shading for about 1 minute to 6 hours under temperature conditions of ice-cold to room temperature.

In one embodiment, the production method according to the second aspect of the present disclosure comprises a step of inducing a myotube from the human urine-derived cell positive for CD90 separated in the separation step, after the separation step (induction step).

The method for inducing human urine-derived cells into myotubes in the induction step is not particularly limited as long as it is a method whereby myotubes can be induced.

In one preferred embodiment, the induction step includes forcibly expressing MYOD1 on human urine-derived cells positive for CD90. The forced expression method is not particularly limited as long as it is a method that those skilled in the art conventionally perform, and for example, it may be to introduce into a cell, a MYOD1 gene, a pre-mRNA, which is a transcription product of a MYOD1 gene, or a messenger RNA (mRNA) obtained by processing of the pre-mRNA.

In one further preferred embodiment, the induction step includes introducing a MYOD1 gene into a human urine-derived cell positive for CD90. A myoblast determination protein 1 (MYOD1) is a muscle-specific transcription factor, one of muscle regulatory factors, and belongs to the MYOD family. It is known that introducing a MYOD1 gene into a fibroblast or the like enables differentiation induction into a myotube. In addition, it is also known that introducing a MYOD1 gene into a human urine-derived cell enables differentiation induction of the human urine-derived cell into a myotube, albeit low efficient and insufficient (Non Patent Literature 2).

MYOD1 genes are well-known in the art and not particularly limited, but preferably a human MYOD1 gene is used. The MYOD1 gene sequence, for example, the human MYOD1 gene sequence, is registered to GenBank under Accession Number NM_002478.4 (by National Center for Biotechnology Information: NCBI).

The introduction of the MYOD1 gene into a human urine-derived cell can be performed by a method known in the art, and for example, it can be performed by the method described in Non Patent Literature 2 or Patent Literature 1. For example, the MYOD1 gene is cloned and incorporated into an appropriate expression vector (e.g., retroviral vector). Besides the MYOD1 gene, a promoter and enhancer, a selection marker gene or the like can be inserted into the expression vector. It is possible to appropriately select a promoter, and it is preferable to use an inducible promoter. For human urine-derived cells, the proliferative capacity thereof is significantly reduced when MYOD1 is expressed to initiate muscle differentiation; however, by using an inducible promoter, it is possible to control the cell growth due to the suppression of muscle differentiation, and the differentiation induction into myotubes. Specifically, the inducible promoter, for example, a TRE3GS promoter, is used to introduce a MYOD1 gene into human urine-derived cells followed by the proliferation of the human urine-derived cell into which the MYOD1 gene has been introduced, and then doxycycline (Dox) is added to a medium to activate the promoter, thereby the MYOD1 gene is expressed to induce the differentiation into myotubes. In addition, even though a selection marker gene is not essential, it can easily select the human urine-derived cell into which the MYOD1 gene has been introduced, and therefore it is preferable to incorporate a selection marker gene into an expression vector. Examples of the selection marker gene include puromycin resistance genes, neomycin resistance genes, Zeocin resistance genes, hygromycin resistance genes, and blasticidin resistance genes. Such an expression vector is introduced into human urine-derived cells, by using a method known in the art, for example, by using a commercially available transfection reagent. The selection of introduced cells is also known in the art, and for example, when inserting puromycin resistance genes into the expression vector, the cells that exhibit resistance to puromycin is selected.

The conditions of introducing a MYOD1 gene in the induction step is not particularly limited, and examples of the medium include a growth medium (obtained by mixing the equivalent amount of REGM Bullet Kit (Lonza; CC-3190) and high-glucose DMEM, and adding thereto 15% tetracycline-free fetal bovine serum, 0.5% Glutamax (Thermo Fisher Scientific, Inc.; 35050-061), 0.5% nonessential amino acid (Thermo Fisher Scientific, Inc.; 11140-050), 2.5 ng/mL fibroblast growth factor-basic (bFGF) (Sigma Aldrich, St. Louis, USA; F0291), PDGF-AB (Peprotech, Rocky Hill, NJ; 100-00AB), EGF (Peprotech; AF-100-15), 1% penicillin/streptomycin, and 0.5 g/mL amphotericin B), a differentiation medium (containing high glucose-containing DMEM with GlutaMax-I (Thermo Fisher Scientific Inc.; 10569-010), 5% horse serum, ITS Liquid Media Supplement (Sigma; 13146), 1 μg/mL doxycycline). In addition, for example, the culturing temperature can be set to a temperature suitable for culturing human urine-derived cells, for example, 30 to 40° C., and preferably about 37° C., and pH is kept, for example, in the vicinity of neutral. The culturing period may be about 1 hour to 4 weeks, and preferably 1 day to 2 weeks.

In one embodiment, the induction step may include exposing the human urine-derived cells positive for CD90 to an epigenetic control compound. Human urine-derived cells are efficiently induced to muscle differentiation by introduction of a MYOD1 gene and exposure to an epigenetic control compound (Patent Literature 1). Therefore, in one embodiment, when the induction step includes exposure to an epigenetic control compound, human urine-derived cells can be more efficiently induced into myotubes. In this case, the introduction of a MYOD1 gene and the exposure to an epigenetic control compound may be simultaneously performed, or the introduction of a MYOD1 gene may be performed after the exposure to an epigenetic control compound.

The epigenetic control refers to the control of gene expression by chromosome changes without involving changes in DNA nucleotide sequences. Examples of such chromosome changes include chemical modifications such as DNA methylation, histone acetylation and methylation in nucleosome, and these chemical modifications of DNA and histone control the gene expression. Therefore, as an inhibitor of enzyme related to such epigenetic control, the epigenetic control compound according to the present disclosure may be an inhibitor of histone methyl transferase (HMT), histone demethylase, histone deacetylase (HDAC), sirtuin 2 (SIRT2) or poly ADP-ribose polymerase (PARP).

Specific examples of the epigenetic control compound and conditions of exposing human urine-derived cells to the epigenetic control compound can include those described in Patent Literature 1 for use.

In one embodiment, the induction step may not include exposing the human urine-derived cells positive for CD90 to an epigenetic control compound. The present inventors have found that the muscle differentiation potential of the human urine-derived cells positive for CD90 is high. According to the production method of one embodiment of the second aspect of the present disclosure, it is possible to efficiently induce human urine-derived cells into myotubes to produce myotubes, though not involving exposure of the human urine-derived cells positive for CD90 to an epigenetic control compound in the induction step. More specifically, in one embodiment, the induction step may not include exposing the human urine-derived cells positive for CD90 to inhibitors which are listed as epigenetic control compounds in Patent Literature 1.

In one embodiment, the induction step may not include producing induced pluripotent stem cells (iPS cells) from human urine-derived cells positive for CD90. In other words, in one embodiment, the induction step may be a step of inducing human urine-derived cells positive for CD90 directly into myotubes (so-called a step of performing direct reprogramming). When producing a somatic cell from another somatic cell, it is possible to produce an iPS cell from the somatic cell after differentiation first, and then the iPS cell is differentiated into another somatic cell; and there has been reported an example of producing an iPS cell from a human urine-derived cell. However, since the muscle differentiation potential of the human urine-derived cells positive for CD90 has been increased, according to the method of the second aspect of the present disclosure, it is possible to induce human urine-derived cells positive for CD90 into myotubes by not including producing an iPS cell from a human urine-derived cell positive for CD90, that is, by a simple method without through an iPS cell once.

In one embodiment, the induction step may include producing induced pluripotent stem cells (iPS cells) from human urine-derived cells positive for CD90. In one embodiment, the induction step may be a step including producing induced pluripotent stem cells (iPS cells) from human urine-derived cells positive for CD90 and producing myotubes from the induced pluripotent stem cells (so-called a step of performing indirect reprogramming).

Success in producing a myotube by the production method according to the second aspect of the present disclosure can be evaluated by, for example, staining the cell nucleus with a nucleus staining reagent such as DAPI or Hoechst and based on the fact that the cell has two or more nuclei. Also, for example, success in producing a myotube can be evaluated based on the fact that a fluorescence image derived from a myofibril is observed when staining the cell with an antibody against a protein that is highly expressed in the myofibril, such as a labeled anti-myosin (MYHC) antibody, or the fact that the cell exhibits strong fluorescence when staining the cell with an antibody against a muscle-specific transcription factor, such as a labeled anti-myogenin antibody. Further, for example, success in producing a myotube can be evaluated based on the fact that the proportion of the cell nuclei contained in the myotube (fusion index) to the cell nuclei in the visual field for the plate-cultured cells is high (e.g., fusion index is twice or more, 3 times or more, or 5 times or more of that before muscle differentiation induction, or fusion index is 0.5 or more, 0.6 or more, or 0.7 or more).

The production method according to the second aspect of the present disclosure can be used for the similar applications to those of the cell population of the human urine-derived cells positive for CD90 according to the first aspect of the present disclosure.

A third aspect of the present disclosure is a cell population of myotubes induced from a cell population of human urine-derived cells positive for CD90. In other words, the third aspect of the present disclosure is a cell population of myotubes produced from a cell population of human urine-derived cells by the production method according to the second aspect. The cell population according to one embodiment of the third aspect of the present disclosure may be a cell population of myotubes induced from a cell population of human urine-derived cells positive for CD90 derived from a muscle disease patient or a muscular dystrophy patient.

A fourth aspect of the present disclosure is a method for separating (or obtaining) a human urine-derived cell of which muscle differentiation potential is high, comprising a step of separating a human urine-derived cell positive for CD90. The human urine-derived cell of which muscle differentiation potential is high according to the fourth aspect of the present disclosure means the cell evaluated that its muscle differentiation potential is high in the cell population of the human urine-derived cells positive for CD90 according to the first aspect of the present disclosure, and may be a cell in which the proportion of the cell nuclei contained in the myotube (fusion index) to the cell nuclei in the visual field when inducing the muscle differentiation under plate-culturing is high (e.g., fusion index is twice or more, 3 times or more, or 5 times or more of that before muscle differentiation induction, or fusion index is 0.5 or more, 0.6 or more, or 0.7 or more). In addition, the step of separating the human urine-derived cells positive for CD90 can be performed by the same method as the separation step in the production method according to the second aspect of the present disclosure, that is, for example, by a flow cytometry method, or an affinity purification or affinity chromatography method. In one embodiment of the forth aspect of the present disclosure, the human urine-derived cell may be a human urine-derived cell derived from a muscle disease patient or a muscular dystrophy patient.

A fifth aspect of the present disclosure is a kit comprising an anti-CD90 antibody or an antigen-binding fragment thereof; and a package insert in which there is described that the human urine-derived cell positive for CD90 is separated as a human urine-derived cell of which muscle differentiation potential is high. According to the kit of the fifth aspect of the present disclosure, it is possible to perform the method for separating the human urine-derived cell of which muscle differentiation potential is high according to the fourth aspect of the present disclosure. In other words, in one embodiment, the fifth aspect of the present disclosure may be a kit to be used in the method for separating the human urine-derived cell of which muscle differentiation potential is high according to the fourth aspect of the present disclosure. Also, in one embodiment, the fifth aspect of the present disclosure may be a kit to be used in the method for separating the human urine-derived cell of which muscle differentiation potential is high according to the second aspect of the present disclosure.

In the present disclosure, an antigen-binding fragment of an anti-CD90 antibody means a part of an antibody that recognizes CD90 and binds thereto, the fragment including a variable domain of the antibody, or at least including an antigen-binding region. Examples of the antigen-binding fragment can include a Fab fragment, a Fab′ fragment, an F(ab′)₂ fragment, and an Fv fragment, and it may be one prepared by a method that those skilled in the art conventionally perform.

The anti-CD90 antibody or an antigen-binding fragment thereof contained in the kit according to one embodiment of the fifth aspect of the present disclosure may be labeled, and it is preferable to be labeled. When the anti-CD90 antibody or an antigen-binding fragment thereof is labeled, it is possible to separate the human urine-derived cell positive for CD90 based on the label used for labeling. The labeling of the anti-CD90 antibody or an antigen-binding fragment thereof may be, for example, labeling by fluorochrome or labeling by affinity tag. Examples of the affinity tag can include biotin, and His tag. When the anti-CD90 antibody or an antigen-binding fragment thereof is labeled by fluorochrome, it is possible to separate human urine-derived cells positive for CD90 by, for example, a flow cytometry method described in the second aspect of the present disclosure. When the anti-CD90 antibody or an antigen-binding fragment thereof is labeled by affinity tag, it is possible to separate human urine-derived cells positive for CD90 by, for example, an affinity purification method or an affinity chromatography method described in the second aspect of the present disclosure.

A sixth aspect of the present disclosure is a method for evaluating muscle differentiation potential of a human urine-derived cell, comprising a step of evaluating the expression of CD90 in the human urine-derived cell. The CD90 expression in the human urine-derived cell can be evaluated by the same method as the evaluation method when evaluating the muscle differentiation potential of the cell included in the cell population of the human urine-derived cells positive for CD90 according to the first aspect of the present disclosure to be high, and for example, it may be evaluated by using, as an index, the fact that a myofibril is formed and/or the fact that the cell nuclei are multinucleated when inducing the muscle differentiation under plate-culturing. In this case, the method for evaluating the CD90 expression in the human urine-derived cell is not particularly limited as long as it is a method whereby the expression of the cell surface protein can be evaluated, and those skilled in the art can freely select a method; and for example, it is possible to evaluate the fluorescence of the human urine-derived cell by a fluorescence microscope, a flow cytometer, a microwell plate reader, a spectrofluorometer or the like, after the human urine-derived cell is brought into contact with the fluorescence-labeled anti-CD90 antibody, or after the human urine-derived cell is brought into contact with the anti-CD90 antibody as a primary antibody and a fluorescence-labeled secondary antibody, and here, the fluorescence intensity can be used as an index. In one embodiment of the sixth aspect of the present disclosure, the human urine-derived cell may be a human urine-derived cell derived from a muscle disease patient or a muscular dystrophy patient.

A seventh aspect of the present disclosure is a kit, comprising an anti-CD90 antibody or an antigen-binding fragment thereof, and a package insert in which there is described that the CD90 expression in a human urine-derived cell is evaluated, and that the human urine-derived cell that is evaluated as the amount of CD90 expressed is high is evaluated as a human urine-derived cell of which muscle differentiation potential is high. According to the kit of the seventh aspect of the present disclosure, it is possible to perform the method for evaluating muscle differentiation potential of a human urine-derived cell according to the sixth aspect of the present disclosure. In other words, in one embodiment, the seventh aspect of the present disclosure may be a kit to be used in the method for evaluating muscle differentiation potential of a human urine-derived cell according to the sixth aspect of the present disclosure. Also, in one embodiment, the seventh aspect of the present disclosure may be a kit to be used in the method for separating the human urine-derived cell of which muscle differentiation potential is high according to the second aspect of the present disclosure. As the anti-CD90 antibody or an antigen-binding fragment thereof according to the seventh aspect of the present disclosure, the ones described in the fifth aspect of the present disclosure can be used.

### EXAMPLES

Hereinafter, the present disclosure will be described in more detail by reference to Examples; however, the present disclosure is not limited to the following Examples.

Note that all experiments shown in Examples were conducted upon approval of National Center of Neurology and Psychiatry (NCNP). In addition, urine provision was conducted at any time with written consent of the person in question or their legally acceptable representative.

### Experimental Method 1: Preparation of Human Urine-Derived Cell

The human urine-derived cells used in Examples of the present disclosure were prepared according to the following protocol. Urine was collected by asking a human subject to urinate into a sterilized plastic bottle (Corning Incorporated, NY, USA; 430281). Primary cells contained in the urine and collected within several hours after urine collection were cultured, by a method that was slightly modified from the method described in Non Patent Literature 4.

Specifically, the collected urine was dispensed in a plurality of 50 ml conical tubes, and centrifuged at 400×g for 10 minutes at room temperature to remove the supernatant. Next, pellets were suspended in PBS followed by collecting into one conical tube, 10 mL of washing liquid (PBS without containing Ca²⁺ and Mg²⁺, containing 1% penicillin/streptomycin (Thermo Fisher Scientific, Inc., Waltham, MA; 15140-122) and 0.5 μg/mL amphotericin B (Sigma Aldrich, St. Louis, USA; A2942)) was added thereto, and the mixture was centrifuged at 200×g for 10 minutes at room temperature to remove the supernatant. To 1.5 mL initial medium (obtained by mixing the equivalent amount of high-glucose DMEM (GE Healthcare, Logan, UT; SH30022.FS) and Ham's F-12 Nutrient Mix (Thermo Fisher Scientific, Inc.; 11765-054), and adding thereto REGM SingleQuots (Lonza, Basel, Switzerland; CC-4127), 10% tetracycline-free fetal bovine serum (Clontech; 631106), 1% penicillin/streptomycin, 0.5 μg/mL amphotericin B), the pellets were suspended and cultured on a gelatin-coated 6-well plate (IWAKI, Shizuoka, Japan; 4810-020) in an incubator with 5% CO₂ at 37° C. The initial medium was added thereto by 1.5 mL every day, and on the fourth day from the culture initiation, the medium was replaced with 2 mL growth medium (obtained by mixing the equivalent amount of REGM Bullet Kit (Lonza; CC-3190) and high-glucose DMEM, and adding thereto 15% tetracycline-free fetal bovine serum, 0.5% Glutamax (Thermo Fisher Scientific, Inc.; 35050-061), 0.5% nonessential amino acid (Thermo Fisher Scientific, Inc.; 11140-050), 2.5 ng/mL fibroblast growth factor-basic (bFGF) (Sigma Aldrich, St. Louis, USA; F0291), PDGF-AB (Peprotech, Rocky Hill, NJ; 100-00AB), EGF (Peprotech; AF-100-15), 1% penicillin/streptomycin, and 0.5 μg/mL amphotericin B, without amphotericin B/gentamicin in REGM Bullet Kit). Human urine-derived cells formed colonies around several days to 2 weeks after the culture initiation. The human urine-derived cells obtained by the above operation were used in Examples of the present disclosure.

### Experimental Method 2: Introduction of MYOD1 Gene into Human Urine-Derived Cell

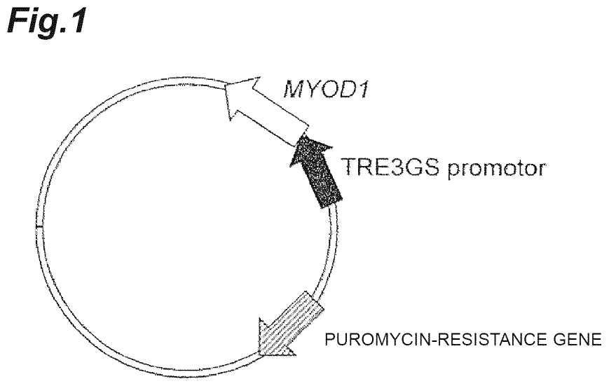

In Examples of the present disclosure, the introduction of a MYOD1 gene into human urine-derived cells was performed according to the following protocol. Note that, for the introduction of a MYOD1 gene into human urine-derived cells, the retroviral vector described in Patent Literature 1 (hereinafter, described as “MYOD1 viral vector”; the schematic diagram is shown in FIG. 1) was used. The MYOD1 viral vector shown in FIG. 1 can induce the expression of the MYOD1 gene by doxycycline (Dox), since the MYOD1 gene is under control of a TRE3GS promoter. In addition, the MYOD1 viral vector includes a puromycin resistance gene as a selection marker.

The MYOD1 viral vector was prepared according to the method described in Patent Literature 1. Human urine-derived cells were seeded (3,000 to 5,000 cells/cm²) on a culture dish or plate, and cultured overnight in the growth medium followed by infection with the MYOD1 viral vector using polybrene to introduce MYOD1 into the human urine-derived cells. 2 days after the infection, puromycin was added to the medium and cultured for 7 days to select the human urine-derived cells into which the MYOD1 gene was introduced.

### [Example 1: Identification of Cell Surface Marker Specific to Human Urine-Derived Cells of which Muscle Differentiation Potential is High, by Single Cell RNAseq Analysis of Human Urine-Derived Cell

According to Experimental Method 1, urine was collected from a healthy subject several times, and human urine-derived cells were established for each collection to obtain a cell population of the human urine-derived cell of each collection. Into each cell population, the MYOD1 gene was introduced according to Experimental Method 2 to induce muscle differentiation. Each cell population thus obtained was immunostained with a mouse anti-MYOGENIN antibody (1:200, Santa Cruz Biotechnology, Inc.; sc-12732) and an anti-MYHC antibody (1:200, eBioscience, #14-6503-82), and the expression levels and fusion indices of MYOGENIN and MYHC were evaluated. The degree of muscle differentiation was evaluated based on the expression levels and fusion indices of MYOGENIN and MYHC to select a cell population of which muscle differentiation potential is high (Good UDCs) and a cell population of which muscle differentiation potential is low (Bad UDCs).

Next, for cell populations of Good UDCs and Bad UDCs before muscle differentiation, as well as muscle-differentiated cell populations 30 days after introduction of MYOD1 gene into Good UDCs and Bad UDCs, single cell RNAseq analysis was conducted. Specifically, cDNA libraries were prepared from the obtained cell population per 1 cell by using a kit from 10×Genomics, Inc., and sequencing was performed on Illumina HiSeq 4000. Using Seurat package (v. 4.0.1), a gene expression profile per 1 cell was analyzed based on a unique molecular identifier (UMI) that was added when preparing libraries, and cells that have similar profiles were categorized as the same cluster. The clustering results were dimensionally compressed by the uniform manifold approximation and projection method (UMAP method), and displayed in two-dimension. Consequently, the cell populations of Good UDCs and Bad UDCs before muscle differentiation, as well as the muscle-differentiated cell populations 30 days after introduction of MYOD1 gene into Good UDCs and Bad UDCs were displayed as different clusters on the gene expression profile.

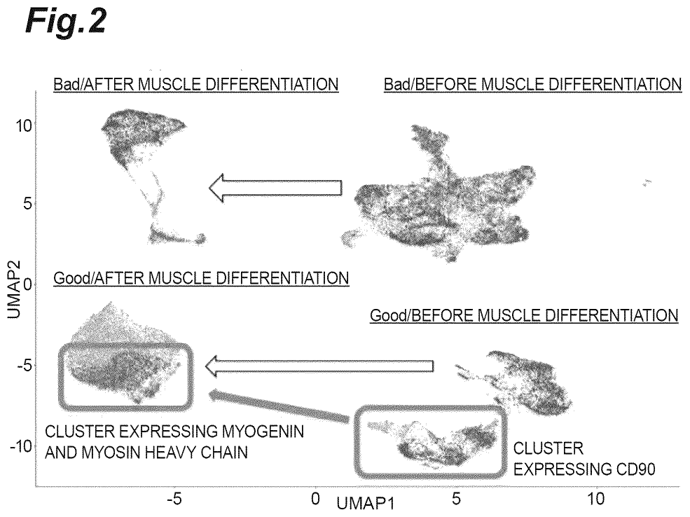

The results are shown in FIG. 2. As shown in FIG. 2, when comparing the Good UDCs clusters and the Bad UDCs clusters, a cluster expressing CD90 only for the Good UDCs was observed. From this, it was suggested that there is a possibility that CD90 is a marker specific to human urine-derived cells of which muscle differentiation potential is high.

### Example 2: Separation of Cell Population of Human Urine-Derived Cells Positive for CD90

Human urine-derived cells were separated by a flow cytometry method using the expression of CD90, which is a candidate for a marker specific to human urine-derived cells of which muscle differentiation potential is high found in Example 1 as an index. The human urine-derived cell obtained from urine collected from a healthy individual according to Experimental Method 1 was exposed to a trypsin-EDTA solution and peeled off from the culture dish, and then suspended in the growth medium. The cell suspension solution thus obtained was centrifuged at 350×g for 5 minutes, the supernatant was removed, and then the resulting product was resuspended in a phosphate buffer solution (PBS) containing 2% fetal bovine serum. To this, a solution of fluorescence-labeled anti-CD90 antibody (FITC anti-human CD90 (Thy1) Antibody, BioLegend, Inc.; 328107) was added to 21-fold dilution, and the mixture was incubated under ice-cold and in a dark place for 20 minutes. The solution in which the human urine-derived cells labeled by the fluorescence-labeled anti-CD90 antibody was suspended was centrifuged at 350×g for 5 minutes, the supernatant was removed, the resulting product was then resuspended in a phosphate buffer solution (PBS) containing 2% fetal bovine serum, and filtered through a 70 μm filter (manufactured by Falcon). The labeled human urine-derived cell thus obtained was separated with a flow cytometer (FACSAria Fusion (BD Biosciences, San Jose, CA)), using the fluorescence intensity of pigment (FITC) modifying the anti-CD90 antibody as an index. At this time, the analysis of data acquired by a flow cytometer was conducted using FlowJo (v10, BD Biosciences).

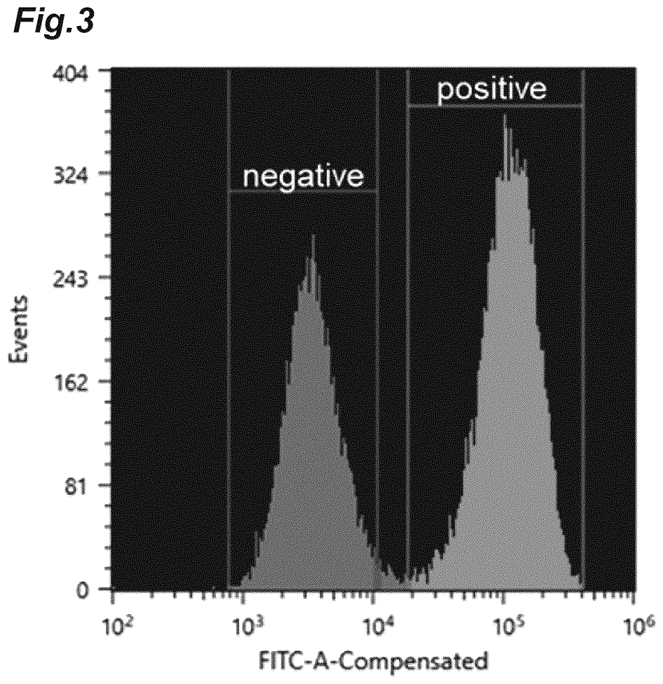

The analysis results by FlowJo are shown in FIG. 3. In the figure, the abscissa shows the fluorescence intensity of pigment (FITC) modifying the anti-CD90 antibody, and the ordinate shows the number of cells. According to FIG. 3, in human urine-derived cells, a cell population well-labeled by the anti-CD90 antibody, that is, a cell population positive for CD90 (Positive), and a cell population less-labeled by the anti-CD90 antibody, that is, a cell population negative for CD90 (Negative) were included. Therefore, as shown in FIG. 3, a total of 2 cell population, which are a cell population positive for CD90 and a cell population negative for CD90, were separated and used for Examples thereafter.

### Example 3: Induction of Muscle Differentiation in Cell Population Positive or Negative for CD90

Into the cell population positive for CD90 and the cell population negative for CD90 which were separated in Example 2, the MYOD1 gene was introduced according to Experimental Method 2. Respective cell populations were expansion-cultured under the conditions of 37° C., 5% CO₂ in a growth medium until the required number of cells for differentiation induction was reached, to induce muscle differentiation. The degree of induction of muscle differentiation in the cell populations after culture was evaluated by immunofluorescent staining and Western blotting.

The immunofluorescent staining was conducted according to the following protocol. Cells contained in the cell population were washed with PBS, then fixed with 4% paraformaldehyde, and added with 0.1% Triton-X to incubate at room temperature for 10 minutes. An anti-myosin heavy chain antibody (1:50, R&D Systems, Inc., Minneapolis, USA; MAB4470), and Alexa Fluor 546 goat anti-mouse IgG (H+L) (1:300, Invitrogen; A11003) were used as a primary antibody and secondary antibody, respectively. For nuclear stain, DAPI was used. It was captured with a fluorescence microscope (BZ-900 or BZ-X800, KEYENCE CORPORATION, Osaka, Japan), and the image was analyzed with BZ-X Analyzer (KEYENCE CORPORATION).

The Western blotting was conducted according to the following protocol. Using a RIPA buffer (Thermo Fisher Scientific Inc.; 89900) containing a protease inhibitor (Roche Diagnostics Corporation, Indianapolis, IN, USA; 04693116001), cells contained in the cell population were dissolved and centrifuged at 4° C., 14,000×g for 15 minutes to collect a supernatant. The total protein concentration was measured with BCA protein assay kit (Thermo Fisher Scientific, Inc.; 23227), degeneration was performed with NuPAGE® LDS Sample Buffer (Thermo Fisher Scientific Inc.; NP0007) and SDS-PAGE was then conducted with NuPAGE (R) Novex Tris-Acetate Gel 3 to 8% (Invitrogen, EA03785BOX) to perform transcription into a PVDF membrane (Millipore. Billerica, MA, USA; IPVH304F0). For antibody reaction, an anti-MYOD1 antibody (1:200; Santa Cruz Biotechnology, Inc.; sc-32758), a mouse anti-MYHC antibody (1:200, eBioscience, #14-6503-82), and a mouse anti-a-tubulin antibody (1:1000, Sigma Aldrich; T6199) were used as primary antibodies, and an anti-mouse horseradish peroxidase-conjugated secondary antibodies (1:50000, Cell Signaling Technology, Inc.) was used as a secondary antibody. After the antibody reaction, bands of interest were detected with ECL Prime Western Blotting Detection Reagent (GE Healthcare, UK; RPN2232).

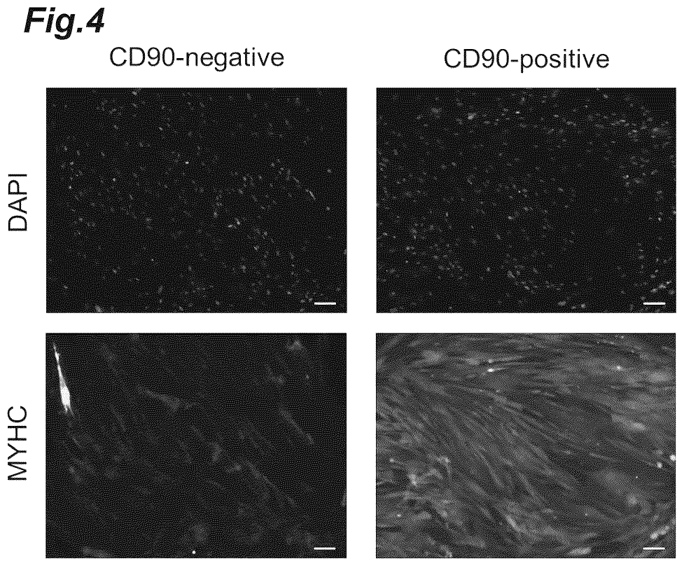

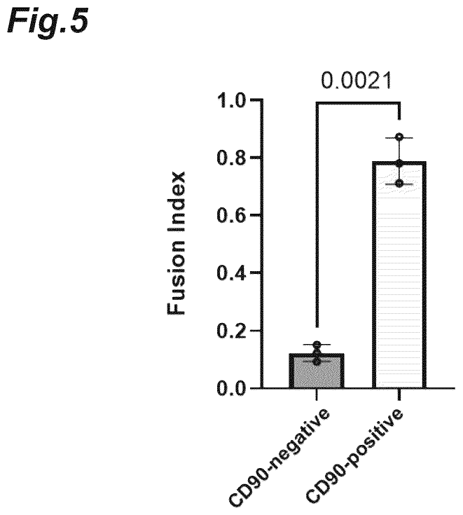

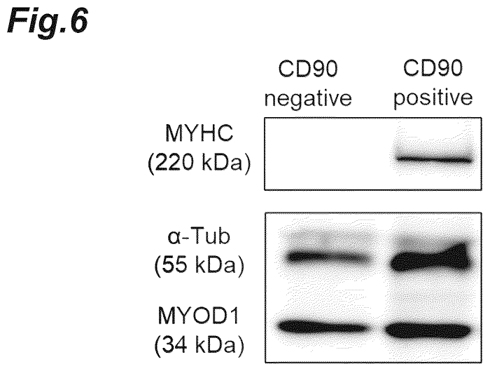

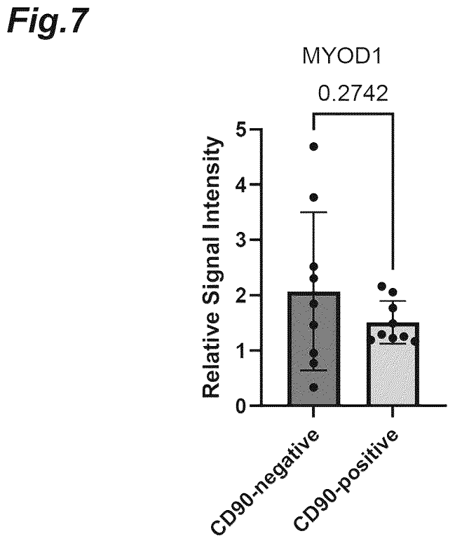

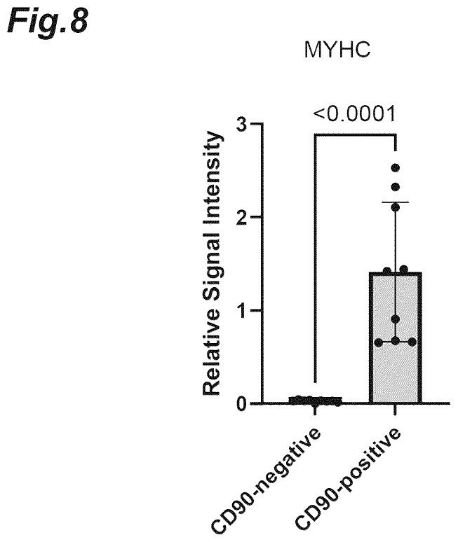

Fluorescence images of immunofluorescent staining are shown in FIG. 4. Fusion index (the proportion of the cell nuclei contained in the myotube shown as MYHC to the cell nuclei shown as DAPI contained in the images) of FIG. 4 is shown in FIG. 5. The results in FIG. 5 are shown as mean±standard deviation (mean±S.D.), and 0.0021 indicates that the p-value in Student t-test was 0.0021. The detection results of bands by Western blotting are shown in FIG. 6. The band intensity of MYOD1 and the band intensity of MYHC standardized with the band intensity of a-tubulin (a-Tub) in FIG. 6 are shown in FIG. 7 and FIG. 8, respectively. The results in FIG. 7 and FIG. 8 are shown as mean±standard deviation (mean±S.D.), and 0.2742 and <0.0001 indicate that the p-values in Student t-test were 0.2742 and less than 0.0001, respectively.

According to the results in FIG. 4 and FIG. 5, many human urine-derived cells were induced into myotubes in the cell population of the human urine-derived cells positive for CD90 (CD90-positive), whereas induction into myotubes was rarely observed in the cell population of the human urine-derived cells negative for CD90 (CD90-negative), revealing that there was significant difference between the muscle differentiation-induced proportions of both cell populations. This was also confirmed by the amount of MYHC expressed from the results of FIG. 6 and FIG. 8.

On the other hand, according to the results of FIG. 6, FIG. 7 and FIG. 8, there is no significant difference in the amount of MYOD1 expressed between positive and negative for CD90, and the significant difference was observed only on the amount of MYHC expressed.

Therefore, the difference in proportions of the muscle differentiation-induced cells between both is not from the difference in introduction efficiency of the MYOD1 gene or expression efficiency of the MYOD1, but revealed that it is from the difference in muscle differentiation induction potential between both.

From the above results, it was indicated that the muscle differentiation potential of cell population of the human urine-derived cell positive for CD90 is high, that is, that CD90 is a cell surface marker specific to human urine-derived cells of which muscle differentiation potential is high.

Note that, as Comparative Example, the cell sorting in the same manner as in Example 2 was performed by using a fluorescence-labeled anti-CD24 antibody (Becton, Dickinson and Company, 560991) in place of the labeled anti-CD90 antibody, and when inducing differentiation by the similar method to that in Example 3, no definite difference in muscle differentiation potential between the cell population positive for CD24 and the cell population negative for CD24 was observed. Also, the cell sorting in the same manner as in Example 2 was performed by using a fluorescence-labeled anti-CD133 antibody (Abcam Limited, ab253259) in place of the labeled anti-CD90 antibody, and when inducing differentiation by the similar method to that in Example 3, no definite difference in muscle differentiation potential between the cell population positive for CD133 and the cell population negative for CD133 was observed, either. Consequently, and also grounded on these results, it was revealed that among various mesenchymal stem cell markers, especially CD90 is a cell surface marker specific to human urine-derived cells of which muscle differentiation potential is high.

### Example 4: Evaluation of Level of Dystrophin Recovery by Exon Skipping Agent, Using Cell Population of Myotube Induced from Cell Population of Human Urine-Derived Cell Positive for CD90 Derived from DMD Patient

Whether the cell population of myotubes induced from the cell population of the human urine-derived cells positive for CD90 reflects abnormality of protein expression in the muscle cells of a muscle disease patient, and whether the evaluation of the exon skipping agent, which is an agent targeting the abnormality of protein expression, by exposure to the agent is possible were examined. Specifically, it was examined, by exposure skipping myotubes produced from urine of a DMD patient to the exon skipping agent, whether the abnormality of dystrophin protein expression in the DMD patient improves, and the expression of dystrophin protein is observed.

Urine was collected from one Duchenne muscular dystrophy patient (DMD patient) having exon 45 deletion in the DMD gene, and establishment and induction of muscle differentiation of the human urine-derived cell were performed according to the procedure described in Examples 1 & 2. In the CD90 (−) group and the CD90 (+) group, in the same manner as in Example 2, the cell population negative for CD90 and the cell population positive for CD90 were each separated. At the seven days after the muscle differentiation induction, in the group with the exon skipping agent, the medium was changed to a differentiation medium (containing high glucose-containing DMEM with GlutaMAX-I (Thermo Fisher Scientific Inc.; 10569-010), 5% horse serum, ITS Liquid Media Supplement (Sigma Aldrich; 13146) and 1 μg/mL doxycycline) containing an exon skipping agent, AON, and 6 μM Endo-Porter (Gene Tools, LLC, Philomath, OR, USA). In the group without the exon skipping agent, the medium was changed to a differentiation medium, instead. At another 3 days later, the medium was changed to a medium with only a differentiation medium, and the cells were recovered at the 14 days after the muscle differentiation induction. The details of used AON were according to Wilton, S. D. et al. Mol Ther 15, 1288-1296 (2007).

The expression of dystrophin protein in the recovered cells was evaluated by Western blotting. Western blotting was performed in the same manner as in Example 3, except that a mouse anti-dystrophin antibody (1:20, Leica Biosystems Nussloch GmbH) and a mouse anti-a-tubulin antibody (1:1000, Sigma Aldrich; T6199) were used as primary antibodies.

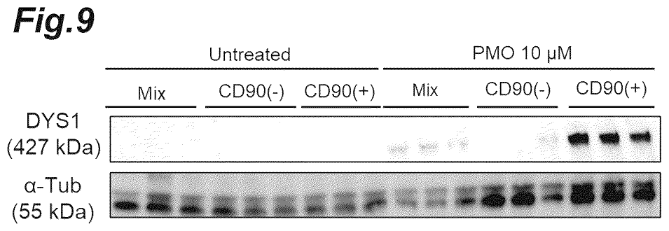

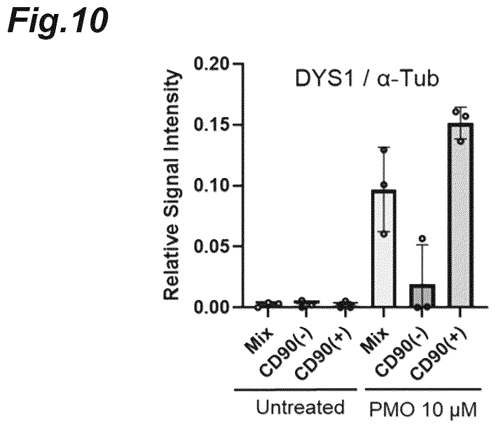

The detection results of bands by Western blotting are shown in FIG. 9. The band intensity of dystrophin 1 (DYS1) standardized with the band intensity of a-tubulin (a-Tub) in FIG. 9 is shown in FIG. 10. In FIG. 9 and FIG. 10, Mix shows the results of the cell population induced to muscle differentiate from human urine-derived cells when separation was not performed using CD90 as an index, and CD90 (−) and CD90 (+) show the results of the cell population induced to muscle differentiate from the cell population negative for CD90 and the cell population of the human urine-derived cells positive for CD90, respectively. In addition, the results in FIG. 10 are shown as mean±standard deviation (mean±S.D.), and PMO 10 μM indicates the group in which the differentiation medium containing 10 μM exon skipping agent was added.

According to FIG. 9 and FIG. 10, the increase in the amount of dystrophin 1 protein expressed by the exon skipping agent can be observed as a band, in the cell population of myotubes obtained by inducing muscle differentiation from the cell population of the human urine-derived cells positive for CD90, and the degree of the increase was larger compared to the case where the cell sorting using CD90 as an index was not performed. From the above, it was revealed that the cell population of myotubes obtained by inducing muscle differentiation from the cell population of the human urine-derived cells positive for CD90 can sharply evaluate the level of dystrophin recovery by the exon skipping agent.

What is claimed is:

1. A cell population of human urine-derived cells positive for CD90.

2. The cell population according to claim 1, wherein the human urine-derived cells positive for CD90 are derived from a muscular disease patient or a muscular dystrophy patient.

3. A method for producing a myotube derived from a human urine-derived cell, comprising:
a step of separating a human urine-derived cell positive for CD90.

4. The method according to claim 3, wherein the step of separating the human urine-derived cell positive for CD90 is a step of separating a human urine-derived cell positive for CD90 from a human urine-derived cell prepared from urine of a muscular disease patient or a muscular dystrophy patient.

5. The method according to claim 3, comprising a step of inducing a myotube from the separated human urine-derived cell positive for CD90 after the step of separating.

6. The method according to claim 4, comprising a step of inducing a myotube from the separated human urine-derived cell positive for CD90 after the step of separating.

7. The method according to claim 5, wherein the step of inducing the myotube comprises introducing a MYOD1 gene into the human urine-derived cell positive for CD90.

8. The method according to claim 6, wherein the step of inducing the myotube comprises introducing a MYOD1 gene into the human urine-derived cell positive for CD90.

9. The method according to claim 5, wherein the step of inducing the myotube does not comprise making an induced pluripotent stem cell from the human urine-derived cell positive for CD90.

10. The method according to claim 6, wherein the step of inducing the myotube does not comprise making an induced pluripotent stem cell from the human urine-derived cell positive for CD90.

11. A cell population of myotubes induced from a cell population of human urine-derived cells positive for CD90.

12. The cell population according to claim 11, wherein the human urine-derived cells positive for CD90 are derived from a muscular disease patient or a muscular dystrophy patient.
